(19) United States
(12) Patent Application Publication
(10) Pub. No.: US 2003/0072807 A1
(43) Pub. Date: Apr. 17, 2003

(54) Solid particulate antifungal compositions for pharmaceutical use

(76) Inventors: Joseph Chung-Tak Wong, Gurnee, IL (US); James E. Kipp, Wauconda, IL (US); Mark J. Doty, Grayslake, IL (US); Christine Rebbeck, Algonquin, IL (US); Pavlos Papadopoulos, Antioch, IL (US)

Correspondence Address: MARK J. BUONAIUTO, ESQ., BAXTER INTERNATIONAL INC., LAW DEPARTMENT, ONE BAXTER PARKWAY, DF3-2E, DEERFIELD, IL, 60015, US

(21) Appl. No.: 10/270,268

(22) Filed: Oct. 11, 2002

Related U.S. Application Data
(63) Continuation-in-part of application No. 10/246,802, filed on Sep. 17, 2002.
Application No. 10/246,802 is a continuation-in-part of application No. 10/035,821, filed on Oct. 19, 2001.
Application No. 10/246,802 is a continuation-in-part of application No. 10/021,692, filed on May 30, 2002.
Application No. 10/021,692 is a continuation-in-part of application No. 09/953,979, filed on Sep. 17, 2001.
Application No. 09/953,979 is a continuation-in-part of application No. 09/874,637, filed on Jun. 5, 2001.
(60) Provisional application No. 60/258,160, filed on Dec. 22, 2000.

(51) Int. Cl. A61K 31/496; A61K 31/4196; A61K 9/16; A61K 9/50

(52) U.S. Cl. 424/490; 514/254.07; 514/383

(57) ABSTRACT

The present invention relates to compositions of submicron-to micron-size particles of antifungal agents. More particularly the invention relates to aqueous suspensions of antifungal agents for pharmaceutical use.

### CROSS REFERENCE TO RELATED APPLICATIONS:

This application is a continuation-in-part of U.S. patent application Ser. No. 10/246,802 filed Sep. 17, 2002 (which is a continuation-in-part of U.S. patent application Ser. No. 10/035,821 filed Oct. 19, 2001), and a continuation-in part of U.S. patent application Ser. No. 10/021,692 filed Dec. 12, 2001, both of which are continuations-in-part of U.S. patent application Ser. No. 09/953,979 filed Sep. 17, 2001, which is a continuation-in-part of U.S. patent application Ser. No. 09/874,637 filed Jun. 5, 2001, which claims priority from provisional Application Serial No. 60/258,160 filed Dec. 22, 2000, all of which are incorporated herein by reference and made a part hereof.

### FEDERALLY SPONSORED RESEARCH OR DEVELOPMENT

Not Applicable.

### BACKGROUND OF THE INVENTION

1. Technical Field

The present invention relates to compositions of antifungal agents. More particularly the invention relates to aqueous suspensions of antifungal agents for pharmaceutical use.

2. Background of the Invention

It is generally recognized that relative to other antimicrobials, there is a profound lack of effective antifungal drugs for the treatment of systemic fungal diseases. Only ten antifungal drugs are approved in the United States for the therapy of systemic fungal infections. The five antifungal drugs which are the most commonly used are amphotericin B, flucytosine, ketoconazole, itraconazole, and fluconazole. The latter three compounds fall under the triazole category with regard to the general molecular structure shown in FIG. 1.

An example of a triazole antifungal agent is itraconazole (FIG. 2). Itraconazole is effective against systemic mycoses, particularly aspergillosis and candidiasis. New oral and intravenous preparations of itraconazole have been prepared in order to overcome bioavailability problems associated with a lack of solubility. For example, the bioavailability of itraconazole is increased when it is formulated in hydroxypropyl-beta-cyclodextrin, a carrier oligosaccharide that forms an inclusion complex with the drug, thereby increasing its aqueous solubility. The commercial preparation is known by the tradename SPORANOX® Injection and was originated by JANSSEN PHARMACEUTICA PRODUCTS, L.P. The drug is currently manufactured by Abbott Labs and distributed by Ortho Biotech, Inc.

Intravenous itraconazole may be useful in selected clinical situations. Examples are achlorhydria in AIDS patients, an inability to effectively absorb oral medications due to concurrent treatments with other drugs, or in critical-care patients who cannot take oral medications. The current commercial product, SPORANOX® Injection, is made available in 25 mL glass vials that contain 250 mg of itraconazole, with 10 g of hydroxypropyl-beta-cyclodextrin (referenced as “HPBCD”). These vials are diluted prior to use in 50 mL of 0.9% saline. The resulting cyclodextrin concentration exceeds 10% (w/v) in the reconstituted product. Although HPBCD has been traditionally regarded as safe for injection, high concentrations, such as 10%, have been reported in animal models to induce significant changes to endothelial tissues (Duncker G.; Reichelt J., Effects of the pharmaceutical cosolvent hydroxypropyl-beta-cyclodextrin on porcine corneal endothelium. Graefe's Archive for Clinical and Experimental Ophthalmology (Germany) 1998, 236/5, 380-389).

Other excipients are often used to formulate poorly water-soluble drugs for intravenous injection. For example, paclitaxel (Taxol®, produced by Bristol-Myers Squibb) contains 52.7% (w/v) of Cremophor® EL (polyoxyethylated castor oil) and 49.7% (v/v) dehydrated alcohol, USP. Administration of Cremophor® EL can lead to undesired hypersensitivity reactions (Volcheck, G. W., Van Dellen, R. G. Anaphylaxis to intravenous cyclosporine and tolerance to oral cyclosporine: case report and review. Annals of Allergy, Asthma, and Immunology, 1998, 80, 159-163; Singla A. K.; Garg A.; Aggarwal D., Paclitaxel and its formulations. International Journal of Pharmaceutics, 2002, 235/1-2, 179-192).

Because of potential toxicity issues associated with solubilizing agents, there is a need for formulations with minimized levels of solubilizer, and in which higher drug loading may be achieved without complete reliance on additives that may cause adverse reactions.

Drugs that are poorly soluble or insoluble in water provide challenges to their delivery. These pharmaceutical agents can have significant benefits when formulated as a stable suspension of submicron- to micron-sized particles. Accurate control of particle size is essential for safe and efficacious use of these formulations. Suitability for pharmaceutical use includes small particle size (<50 μm), low toxicity (as from toxic formulation components or residual solvents), and bioavailability of the drug particles after administration.

One approach to delivering an insoluble drug is disclosed in U.S. Pat. No. 2,745,785. This patent discloses a method for preparing crystals of penicillin G suitable for parenteral administration. The method includes the step of recrystallizing the penicillin G from a formamide solution by adding water to reduce the solubility of the penicillin G. The '785 Patent further provides that the penicillin G particles can be coated with wetting agents such as lecithin, or emulsifiers, surface-active and defoaming agents, or partial higher fatty acid esters of sorbitan or polyoxyalkyklene derivatives thereof, or aryl alkyl polyether alcohols or salts thereof. The '785 patent further discloses micronizing the penicillin G with an air blast under pressure to form crystals ranging from about 5 to 20 microns.

Another approach is disclosed in U.S. Pat. No. 5,118,528 which discloses a process for preparing nanoparticles. The process includes the steps of: (1) preparing a liquid phase of a substance in a solvent or a mixture of solvents to which may be added one or more surfactants; (2) preparing a second liquid phase of a non-solvent or a mixture of non-solvents, the non-solvent is miscible with the solvent or mixture of solvents for the substance; (3) adding together the solutions of (1) and (2) with stirring; and (4) removing of unwanted solvents to produce a colloidal suspension of nanoparticles. The '528 Patent discloses that it produces particles of the substance smaller than 500 nm without the supply of energy. In particular the '528 Patent states that it is undesirable to use high energy equipment such as sonicators and homogenizers.

U.S. Pat. No. 4,826,689 discloses a method for making uniformly sized particles from water-insoluble drugs or other organic compounds. First, a suitable solid organic compound is dissolved in an organic solvent, and the solution can be diluted with a non-solvent. Then, an aqueous precipitating liquid is infused, precipitating non-aggregated particles with substantially uniform mean diameter. The particles are then separated from the organic solvent. Depending on the organic compound and the desired particle size, the parameters of temperature, ratio of non-solvent to organic solvent, infusion rate, stir rate, and volume can be varied according to the invention. The '689 Patent discloses this process forms a drug in a metastable state which is thermodynamically unstable and which eventually converts to a more stable crystalline state. The '689 Patent discloses trapping the drug in a metastable state in which the free energy lies between that of the starting drug solution and the stable crystalline form. The '689 Patent discloses utilizing crystallization inhibitors (e.g., polyvinylpyrrolidinone) and surface-active agents (e.g., poly(oxyethylene)-co-(oxypropylene) ) to render the precipitate stable enough to be isolated by centrifugation, membrane filtration or reverse osmosis.

In U.S. Pat. Nos. 5,091,188; 5,091,187 and 4,725,442 which disclose (a) either coating small drug particles with natural or synthetic phospholipids or (b) dissolving the drug in a suitable lipophilic carrier and forming an emulsion stabilized with natural or semisynthetic phospholipids.

Another approach to providing insoluble drugs for pharmaceutical use is disclosed in U.S. Pat. No. 5,145,684. The '684 Patent discloses the wet milling of an insoluble drug in the presence of a surface modifier to provide a drug particle having an average effective particle size of less than 400 nm. The '684 Patent emphasizes the desirability of not using any solvents in its process. The '684 Patent discloses the surface modifier is adsorbed on the surface of the drug particle in an amount sufficient to prevent agglomeration into larger particles.

Yet another attempt to provide insoluble drugs for pharmaceutical use is disclosed in U.S. Pat. No. 5,922,355. The '355 Patent discloses providing submicron sized particles of insoluble drugs using a combination of surface modifiers and a phospholipid followed by particle size reduction using techniques such as sonication, homogenization, milling, microfluidization, precipitation or recrystallization.

U.S. Pat. No. 5,780,062 discloses a method of preparing small particles of insoluble drugs by (1) dissolving the drug in a water-miscible first solvent; (2) preparing a second solution of a polymer and an amphiphile in an aqueous second solvent in which the drug is substantially insoluble whereby a polymer/amphiphile complex is formed; and (3) mixing the solutions from the first and second steps to precipitate an aggregate of the drug and polymer/amphiphile complex.

U.S. Pat. No. 5,858,410 discloses a pharmaceutical nanosuspension suitable for pharmaceutical use. The '410 patent discloses subjecting at least one solid therapeutically active compound dispersed in a solvent to high pressure homogenization in a piston-gap homogenizer to form particles having an average diameter, determined by photon correlation spectroscopy (PCS) of 10 nm to 1000 nm, the proportion of particles larger than 5 μm in the total population being less than 0.1% (number distribution determined with a Coulter counter), without prior conversion into a melt, wherein the active compound is solid at room temperature and is insoluble, only sparingly soluble or moderately soluble in water, aqueous media and/or organic solvents. The Examples in the '410 Patent disclose jet milling prior to homogenization.

U.S. Pat. No. 4,997,454 discloses a method for making uniformly sized particles from solid compounds. The method of the '454 Patent includes the steps of dissolving the solid compound in a suitable solvent followed by infusing precipitating liquid thereby precipitating non-aggregated particles with substantially uniform mean diameter. The particles are then separated from the solvent. The '454 Patent discourages forming particles in a crystalline state because during the precipitating procedure the crystal can dissolve and recrystallize thereby broadening the particle size distribution range. The '454 Patent encourages during the precipitating procedure to trap the particles in a metastable particle state.

U.S. Pat. No. 5,605,785 discloses a process for forming amorphous dispersions of photographically useful compounds. The process of forming amorphous dispersions include any known process of emulsification that produces a disperse phase having amorphous particulates.

U.S. Pat. No. 6,245,349 discloses concentrated drug delivery compositions of antifungal agents formulated with a phospholipid component, a component selected from propylene glycol or certain polyethylene glycol compounds, a high hydrophilic-lipophilic balance (HLB) surfactant, and the drug component, with water and/or an oil component optional. The concentrated drug delivery compositions can be diluted with an aqueous fluid to form an oil-in-water microemulsion composition.

### SUMMARY OF THE INVENTION

The present invention relates to compositions of an aqueous suspension of submicron-to micron-size particles of an antifungal agent coated with one or more surfactants. The particles of the antifungal agent should have a volume-weighted mean particle size of less than about 50 μm in diameter as determined by light scattering (HORIBA) or by microscopic measurements. More preferably the particles should be less than about 7 μm, even more preferably less than about 2 μm and even more preferably less than about 400 nm and most preferably less than about 100 nm or any range or combination of ranges therein.

In an embodiment of the invention, the antifungal agent is a triazole antifungal agent. In another embodiment of the invention, the triazole antifungal agent is selected from itraconazole, ketoconazole, miconazole, fluconazole, ravuconazole, voriconazole, saperconazole, eberconazole, genaconazole, and posaconazole. In a preferred embodiment of the invention, the antifungal agent is itraconazole.

In a preferred embodiment, the composition is suitable for pharmaceutical use.

Suitable surfactants for coating the particles in the present invention can be selected from ionic surfactants, nonionic surfactants, biologically derived surfactants, or amino acids and their derivatives.

A preferred ionic surfactant is a bile salt, and a preferred bile salt is deoxycholate. A preferred nonionic surfactant is a polyalkoxyether, and a preferred polyalkoxyether is Poloxamer 188. Another preferred nonionic surfactant is Solutol HS 15 (polyethylene-660-hydroxystearate). Still yet another preferred nonionic surfactant is hydroxyethylstarch. A preferred biologically derived surfactant is albumin.

In one preferred embodiment, the particles of the present invention are suspended in an aqueous medium further having a pH adjusting agent. Suitable pH adjusting agents include, but are not limited to, tris buffer, phosphate, acetate, lactate, THAM (tris(hydroxymethyl)aminomethane), meglumine (N-methylglucosamine), citrate, sodium hydroxide, hydrochloric acid, and amino acids such as glycine, arginine, lysine, alanine and leucine. The aqueous medium may also include an osmotic pressure adjusting agent, such as but not limited to glycerin, a monosaccharide such as dextrose, and sugar alcohols such as mannitol and sorbitol.

In another embodiment of the present invention, the antifungal agent is present in an amount preferably from about 0.01% to about 50% weight to volume (w/v), more preferably from about 0.05% to about 30% w/v, and most preferably from about 0.1% to about 20% w/v.

In yet another embodiment, the surfactants are present in an amount of preferably from about 0.001% to about 5% w/v, more preferably from about 0.005% to about 5%, and most preferably from about 0.01% to about 5% w/v.

In an embodiment of the present invention, the aqueous medium of the composition is removed to form dry particles, which may then be reformulated to an acceptable pharmaceutical dosage form.

In another embodiment, the aqueous suspension composition is frozen.

In a preferred embodiment of the present invention, the composition comprises an aqueous suspension of submicron-to micron-size particles of itraconazole present at 0.01 to 50% w/v, the particles are coated with 0.001 to 5% w/v of a bile salt (e.g., deoxycholate) and 0.001 to 5% w/v polyalkoxyether (for example, Poloxamer 188), and glycerin added to adjust osmotic pressure of the formulation.

In another preferred embodiment of the present invention, the composition comprises an aqueous suspension of itraconazole present at about 0.01 to 50% w/v, the particles coated with about 0.001 to 5% w/v of a bile salt (for example, deoxycholate), and 0.001 to 5% polyethylene-660-hydroxystearate (w/v), and glycerin added to adjust osmotic pressure of the formulation.

In another preferred embodiment of the present invention, the composition comprises an aqueous suspension of itraconazole present at about 0.01 to 50% w/v, the particles are coated with about 0.001 to 5% of polyethylene-660-hydroxystearate (w/v), and glycerin added to adjust osmotic pressure of the formulation.

In still yet another preferred embodiment of the present invention, the composition comprises an aqueous suspension of itraconazole present at 0.01 to 50% w/v, the particles are coated with about 0.001 to 5% albumin (w/v).

In a further preferred embodiment, the composition of the present invention is prepared by a microprecipitation method which includes the steps of: (i) dissolving in the antifungal agent in a first water-miscible first solvent to form a solution; (ii) mixing the solution with a second solvent which is aqueous to define a pre-suspension; and (iii) adding energy to the pre-suspension to form particles having an average effective particle size of less than 50 μm; more preferably less than about 7 μm, even more preferably less than about 2 μm, and even more preferably less than about 400 nm, and most preferably less than about 100 nm or any range or combination of ranges therein, wherein the solubility of the antifungal agent is greater in the first solvent than in the second solvent, and the first solvent or the second solvent comprising one or more surfactants selected from the group consisting of: nonionic surfactants, ionic surfactants, biologically derived surfactants, and amino acids and their derivatives.

These and other aspects and attributes of the present invention will be discussed with reference to the following drawings and accompanying specification.

### BRIEF DESCRIPTION OF THE DRAWINGS:

FIG. 1 is the general molecular structure of a triazole antifungal agent;

FIG. 2 is the molecular structure of itraconazole;

FIG. 3 is a schematic diagram of Method A of the microprecipitation process used in the present invention to prepare the suspension;

FIG. 4 is a schematic diagram of Method B of the microprecipitation process used in the present invention to prepare the suspension;

FIG. 5 is a graph comparing the pharmacokinetics of SPORANOX® with Formulation 1 suspension of itraconazole of the present invention, wherein ITC=plasma concentration of itraconazole measured after bolus injection of Formulation 1 (80 mg/kg), ITC−OH=plasma concentration of primary metabolite, hydroxyitraconazole, measured after bolus injection of Formulation 1 (80 mg/kg), Total=combined concentration of itraconazole and hydroxyitraconazole (ITC+ITC−OH) measured after bolus injection of Formulation 1 (80 mg/kg), Sporanox−ITC=plasma concentration of itraconazole measured after bolus injection of 20 mg/kg Sporanox IV, Sporanox−ITC−OH=plasma concentration of primary metabolite, hydroxyitraconazole, measured after bolus injection of 20 mg/kg Sporanox IV, Sporanox−Total=combined concentration of itraconazole and hydroxyitraconazole (ITC+ITC−OH) measured after bolus injection of 20 mg/kg Sporanox IV;

FIG. 6 is a graph comparing the mean body weight and C. albicans colony count data for treatments with SPORANOX® (top panel) and Formulation 1 (bottom panel);

FIG. 7 is a graph showing the distribution of itraconazole (1−ITC) and its metabolite hydroxy-itraconazole (1−ITC−OH) in the kidney after the administration of various doses of suspension formulation (Formulation 1) of itraconazole (numbers beside each data point denote fungal colony counts found in the kidney associated with the suspension dose represented by the data point); and

FIG. 8 is a graph showing the fungal counts in the kidney which decrease with rising kidney itraconazole levels. (Key: S=SPORANOX, N=Formulation 1 nanosuspension).

### DETAILED DESCRIPTION OF THE PREFERRED EMBODIMENT

While this invention is susceptible of embodiment in many different forms, there is shown in the drawing, and will be described herein in detail, specific embodiments thereof with the understanding that the present disclosure is to be considered as an exemplification of the principles of the invention and is not intended to limit the invention to the specific embodiments illustrated.

The present invention relates to an antifungal composition comprising an aqueous suspension of submicron- to micron-size particles of the antifungal agent coated with one or more surfactants. The particles of the antifungal agent should have a volume-weighted particle size of less than about 50 μm in diameter as determined by light scattering (HORIBA), or by microscopic measurements. More preferably the particles should be less than about 7 μm, more preferably less than about 2 μm, even more preferably less than about 400 nm, and even more preferably less than about 200 nm and most preferably less than about 100 nm or any range or combination of ranges therein.

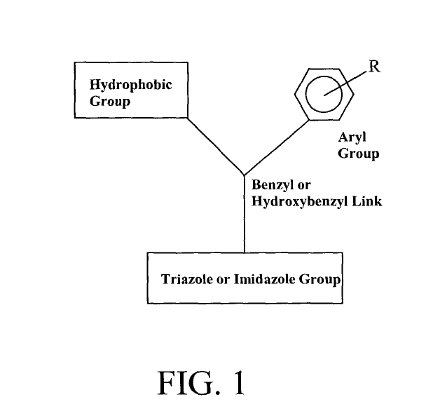

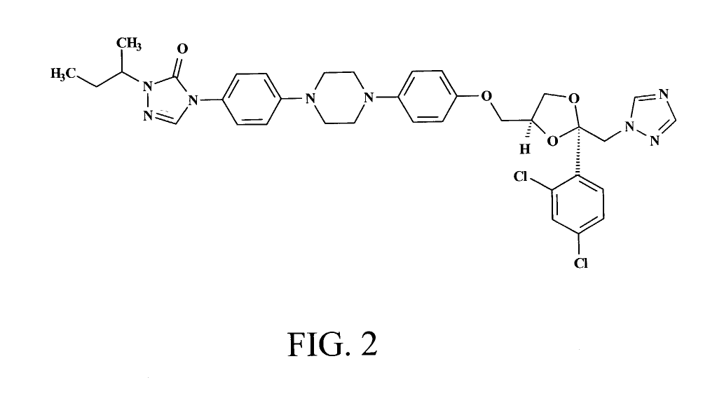

The antifungal agent is preferably a poorly water soluble organic compound. What is meant by “poorly water soluble” is that the water solubility of the compound is less than 10 mg/ml, and preferably, less than 1 mg/ml. A preferred class of antifungal agent is the triazole antifungal agents having a general molecular structure as shown in FIG. 1. Examples of triazole antifungal agents include, but are not limited to: itraconazole, ketoconazole, miconazole, fluconazole, ravuconazole, voriconazole, saperconazole, eberconazole, genaconazole, and posaconazole. A preferred antifungal agent for the present invention is itraconazole. The molecular structure of itraconazole is shown in FIG. 2.

The present invention is suitable for pharmaceutical use. The compositions can be administered by various routes. Preferred routes of administration are parenteral and oral. Modes of parenteral administration include intravenous, intra-arterial, intrathecal, intraperitoneal, intraocular, intra-articular, intramuscular, subcutaneous injection, and the like. The present invention may also be administered via other routes that include oral, buccal, periodontal, rectal, nasal, pulmonary, transdermal, or topical. In an embodiment of the present invention, the aqueous medium of the composition is removed to form dry particles. The method to remove the aqueous medium can be any method known in the art. One example is evaporation. Another example is freeze drying or lyophilization. The dry particles may then be formulated into any acceptable physical form including, but is not limited to, solutions, tablets, capsules, suspensions, creams, lotions, emulsions, aerosols, powders, incorporation into reservoir or matrix devices for sustained release (such as implants or transdermal patches), and the like. Administration routes of these pharmaceutical forms include, but are not limited to parenteral, oral, buccal, periodontal, rectal, nasal, pulmonary, transdermal and topical. Furthermore, the active pharmaceutical agent may be delivered using controlled or sustained release formulations, incorporation into delivery devices such as implantable devices and transdermal patches. Drug may formulated for systemic delivery or for tissue- and/or receptor-specific targeting.

The aqueous suspension of the present invention may also be frozen to improve stability upon storage. Freezing of an aqueous suspension to improve stability is disclosed in the commonly assigned and co-pending U.S. patent application Ser. No. 60/347,548, which is incorporated herein by reference and made a part hereof.

In an embodiment of the present invention, the antifungal agent is present in an amount preferably from about 0.01% to about 50% weight to volume (w/v), more preferably from about 0.05% to about 30% w/v, and most preferably from about 0.1% to about 20% w/v.

Suitable surfactants for coating the particles in the present invention can be selected from ionic surfactants, nonionic surfactants, biologically derived surfactants or amino acids and their derivatives. Ionic surfactants can be anionic or cationic.

Suitable anionic surfactants include but are not limited to: potassium laurate, sodium lauryl sulfate, sodium dodecylsulfate, alkyl polyoxyethylene sulfates, sodium alginate, dioctyl sodium sulfosuccinate, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable cationic surfactants include but are not limited to quaternary ammonium compounds, such as benzalkonium chloride, cetyltrimethylammonium bromide, lauryldimethylbenzylammonium chloride, acyl carnitine hydrochlorides, or alkyl pyridinium halides.

Suitable nonionic surfactants include: polyoxyethylene fatty alcohol ethers (Macrogol and Brij), polyoxyethylene sorbitan fatty acid esters (Polysorbates), polyoxyethylene fatty acid esters (Myrj), sorbitan esters (Span), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxomers), polaxamines, methylcellulose, hydroxycellulose, hydroxy propylcellulose, hydroxy propylmethylcellulose, noncrystalline cellulose, polysaccharides including starch and starch derivatives such as hydroxyethylstarch (HES), polyvinyl alcohol, and polyvinylpyrrolidone. In a preferred form of the invention, the nonionic surfactant is a polyoxyethylene and polyoxypropylene copolymer and preferably a block copolymer of propylene glycol and ethylene glycol. Such polymers are sold under the tradename POLOXAMER also sometimes referred to as PLURONIC®, and sold by several suppliers including Spectrum Chemical and Ruger. Among polyoxyethylene fatty acid esters is included those having short alkyl chains. One example of such a surfactant is SOLUTOL® HS 15, polyethylene-660-hydroxystearate, manufactured by BASF Aktiengesellschaft.

Suitable biologically derived surfactants include such molecules as albumin, casein, heparin, hirudin or other appropriate proteins or polysaccharides. Other suitable surfactants include any amino acids such as leucine, alanine, valine, isoleucine, lysine, aspartic acid, glutamic acid, methionine, phenylalanine, or any derivatives of these amino acids such as, for example, amide or ester derivatives and polypeptides formed from these amino acids.

A preferred ionic surfactant is a bile salt, and a preferred bile salt is deoxycholate. A preferred nonionic surfactant is a polyalkoxyether, and a preferred polyalkoxyether is Poloxamer 188. Another preferred nonionic surfactant is Solutol HS 15 (polyethylene-660-hydroxystearate). Still yet another preferred nonionic surfactant is hydroxyethylstarch. A preferred biologically derived surfactant is albumin.

In another embodiment of the present invention, the surfactants are present in an amount of preferably from about 0.001% to 5% w/v, more preferably from about 0.005% to about 5% w/v, and most preferably from about 0.01% to 5% w/v.

In a preferred embodiment of the present invention, the particles are suspended in an aqueous medium further including a pH adjusting agent. Suitable pH adjusting agents include, but are not limited to, tris buffer, phosphate, acetate, lactate, THAM (tris(hydroxymethyl)aminomethane), meglumine (N-methylglucosamine), citrate, sodium hydroxide, hydrochloric acid, and amino acids such as glycine, arginine, lysine, alanine and leucine. The aqueous medium may additionally include an osmotic pressure adjusting agent, such as but not limited to glycerin, a monosaccharide such as dextrose, and sugar alcohols such as mannitol and sorbitol.

In a preferred embodiment of the present invention, the composition comprises an aqueous suspension of particles of itraconazole present at 0.01 to 50% w/v, the particles are coated with 0.001 to 5% w/v of a bile salt (e.g., deoxycholate) and 0.001 to 5% w/v polyalkoxyether (for example, Poloxamer 188), and glycerin added to adjust osmotic pressure of the formulation.

In another preferred embodiment of the present invention, the composition comprises an aqueous suspension of particles of itraconazole present at about 0.01 to 50% w/v, the particles coated with about 0.001 to 5% w/v of a bile salt (for example, deoxycholate) and 0.001 to 5% polyethylene-660-hydroxystearate w/v, and glycerin added to adjust osmotic pressure of the formulation.

In another preferred embodiment of the present invention, the composition comprises an aqueous suspension of itraconazole present at about 0.01 to 50% w/v, the particles are coated with about 0.001 to 5% of polyethylene-660-hydroxystearate w/v, and glycerin added to adjust osmotic pressure of the formulation.

In still yet another preferred embodiment of the present invention, the composition comprises an aqueous suspension of itraconazole present at 0.01 to 50% w/v, the particles are coated with about 0.001 to 5% albumin w/v.

The method for preparing the suspension in the present invention is disclosed in commonly assigned and co-pending U.S. patent applications Ser. Nos. 09/874,499; 09/874,799; 09/874,637; and 10/021,692; which are incorporated herein by reference and made a part hereof. A general procedure for preparing the suspension useful in the practice of this invention follows.

The processes can be separated into three general categories. Each of the categories of processes share the steps of: (1) dissolving an antifungal agent in a water miscible first organic solvent to create a first solution; (2) mixing the first solution with a second solvent of water to precipitate the antifungal agent to create a pre-suspension; and (3) adding energy to the presuspension in the form of high-shear mixing or heat to provide a stable form of the antifungal agent having the desired size ranges defined above.

The three categories of processes are distinguished based upon the physical properties of the antifungal agent as determined through x-ray diffraction studies, differential scanning calorimetry (DSC) studies or other suitable study conducted prior to the energy-addition step and after the energy-addition step. In the first process category, prior to the energy-addition step the antifungal agent in the presuspension takes an amorphous form, a semi-crystalline form or a supercooled liquid form and has an average effective particle size. After the energy-addition step, the antifungal agent is in a crystalline form having an average effective particle size essentially the same as that of the presuspension (i.e., from less than about 50 μm).

In the second process category, prior to the energy-addition step the antifungal agent is in a crystalline form and has an average effective particle size. After the energy-addition step, the antifungal agent is in a crystalline form having essentially the same average effective particle size as prior to the energy-addition step but the crystals after the energy-addition step are less likely to aggregate.

The lower tendency of the organic compound to aggregate is observed by laser dynamic light scattering and light microscopy.

In the third process category, prior to the energy-addition step the antifungal agent is in a crystalline form that is friable and has an average effective particle size. What is meant by the term “friable” is that the particles are fragile and are more easily broken down into smaller particles. After the energy-addition step the organic compound is in a crystalline form having an average effective particle size smaller than the crystals of the pre-suspension. By taking the steps necessary to place the organic compound in a crystalline form that is friable, the subsequent energy-addition step can be carried out more quickly and efficiently when compared to an organic compound in a less friable crystalline morphology.

The energy-addition step can be carried out in any fashion wherein the pre-suspension is exposed to cavitation, shearing or impact forces. In one preferred form of the invention, the energy-addition step is an annealing step. Annealing is defined in this invention as the process of converting matter that is thermodynamically unstable into a more stable form by single or repeated application of energy (direct heat or mechanical stress), followed by thermal relaxation. This lowering of energy may be achieved by conversion of the solid form from a less ordered to a more ordered lattice structure. Alternatively, this stabilization may occur by a reordering of the surfactant molecules at the solid-liquid interface.

These three process categories will be discussed separately below. It should be understood, however, that the process conditions such as choice of surfactants or combination of surfactants, amount of surfactant used, temperature of reaction, rate of mixing of solutions, rate of precipitation and the like can be selected to allow for any drug to be processed under any one of the categories discussed next.

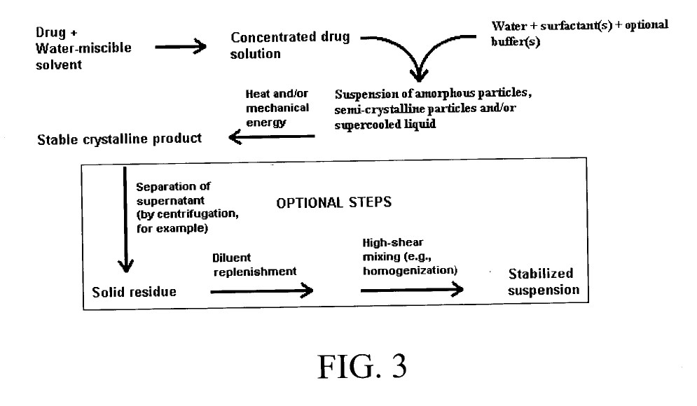

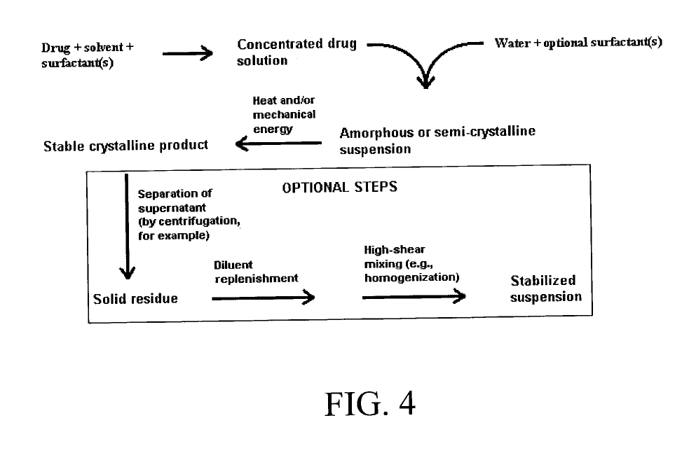

The first process category, as well as the second and third process categories, can be further divided into two subcategories, Method A, and B shown diagrammatically in FIG. 3 and FIG. 4, respectively.

The first solvent according to the present invention is a solvent or mixture of solvents in which the antifungal agent of interest is relatively soluble and which is miscible with the second solvent. Examples of such solvents include, but are not limited to: polyvinylpyrrolidone, N-methyl-2-pyrrolidinone (also called N-methyl-2-pyrrolidone), 2-pyrrolidone, dimethyl sulfoxide, dimethylacetamide, lactic acid, methanol, ethanol, isopropanol, 3-pentanol, n-propanol, glycerol, butylene glycol (butanediol), ethylene glycol, propylene glycol, mono- and diacylated monoglycerides (such as glyceryl caprylate), dimethyl isosorbide, acetone, dimethylformamide, 1,4-dioxane, polyethylene glycol (for example, PEG-4, PEG-8, PEG-9, PEG-12, PEG-14, PEG-16, PEG-120, PEG-75, PEG-150), polyethylene glycol esters (examples such as PEG-4 dilaurate, PEG-20 dilaurate, PEG-6 isostearate, PEG-8 palmitostearate, PEG-150 palmitostearate), polyethylene glycol sorbitans (such as PEG-20 sorbitan isostearate), polyethylene glycol monoalkyl ethers (examples such as PEG-3 dimethyl ether, PEG-4 dimethyl ether), polypropylene glycol (PPG), polypropylene alginate, PPG-10 butanediol, PPG-10 methyl glucose ether, PPG-20 methyl glucose ether, PPG-15 stearyl ether, propylene glycol dicaprylate/dicaprate, propylene glycol laurate.

Method A

In Method A (see FIG. 3), the antifungal agent is first dissolved in the first solvent to create a first solution. The antifungal agent can be added from about 0.01% (w/v) to about 50% (w/v) depending on the solubility of the antifungal agent in the first solvent. Heating of the concentrate from about 30° C. to about 100° C. may be necessary to ensure total dissolution of the antifungal agent in the first solvent.

A second aqueous solution is provided with one or more surfactants added thereto. The surfactants can be selected from an ionic surfactant, a nonionic surfactant or a biologically derived surfactant set forth above.

It may also be desirable to add a pH adjusting agent to the second solution such as sodium hydroxide, hydrochloric acid, tris buffer or citrate, acetate, lactate, meglumine, or the like. The second solution should have a pH within the range of from about 3 to about 11.

In a preferred form of the invention, the method for preparing submicron sized particles of an antifungal agent includes the steps of adding the first solution to the second solution. The addition rate is dependent on the batch size, and precipitation kinetics for the antifungal agent. Typically, for a small-scale laboratory process (preparation of 1 liter), the addition rate is from about 0.05 cc per minute to about 10 cc per minute. During the addition, the solutions should be under constant agitation. It has been observed using light microscopy that amorphous particles, semi-crystalline solids, or a supercooled liquid are formed to create a pre-suspension. The method further includes the step of subjecting the pre-suspension to an annealing step to convert the amorphous particles, supercooled liquid or semicrystalline solid to a crystalline more stable solid state. The resulting particles will have an average effective particles size as measured by dynamic light scattering methods (e.g., photocorrelation spectroscopy, laser diffraction, low-angle laser light scattering (LALLS), medium-angle laser light scattering (MALLS), light obscuration methods (Coulter method, for example), rheology, or microscopy (light or electron) within the ranges set forth above).

The energy-addition step involves adding energy through sonication, homogenization, counter current flow homogenization (e.g., the Mini DeBEE 2000 homogenizer, available from BEE Incorporated, N.C., in which a jet of fluid is directed along a first path, and a structure is interposed in the first path to cause the fluid to be redirected in a controlled flow path along a new path to cause emulsification or mixing of the fluid), microfluidization, or other methods of providing impact, shear or cavitation forces. The sample may be cooled or heated during this stage. In one preferred form of the invention the annealing step is effected by homogenization. In another preferred form of the invention the annealing may be accomplished by ultrasonication. In yet another preferred form of the invention the annealing may be accomplished by use of an emulsification apparatus as described in U.S. Pat. No. 5,720,551 which is incorporated herein by reference and made a part hereof.

Depending upon the rate of annealing, it may be desirable to adjust the temperature of the processed sample to within the range of from approximately −30° C. to 30° C. Alternatively, in order to effect a desired phase change in the processed solid, it may also be necessary to heat the pre-suspension to a temperature within the range of from about 30° C. to about 100° C. during the annealing step.

Method B

Method B differs from Method A in the following respects. The first difference is a surfactant or combination of surfactants are added to the first solution. The surfactants may be selected from ionic surfactants, nonionic surfactants, or biologically derived as set forth above.

A drug suspension resulting from application of the processes described in this invention may be administered directly as an injectable solution, provided Water for Injection is used in formulation and an appropriate means for solution sterilization is applied. Sterilization may be accomplished by separate sterilization of the drug concentrate (drug, solvent, and optional surfactant) and the diluent medium (water, and optional buffers and surfactants) prior to mixing to form the pre-suspension. Sterilization methods include pre-filtration first through a 3.0 micron filter followed by filtration through a 0.45-micron particle filter, followed by steam or heat sterilization or sterile filtration through two redundant 0.2-micron membrane filters.

Optionally, a solvent-free suspension may be produced by solvent removal after precipitation. This can be accomplished by centrifugation, dialysis, diafiltration, force-field fractionation, high-pressure filtration or other separation techniques well known in the art. Complete removal of N-methyl-2-pyrrolidinone was typically carried out by one to three successive centrifugation runs; after each centrifugation the supernatant was decanted and discarded. A fresh volume of the suspension vehicle without the organic solvent was added to the remaining solids and the mixture was dispersed by homogenization. It will be recognized by others skilled in the art that other high-shear mixing techniques could be applied in this reconstitution step.

Furthermore, any undesired excipients such as surfactants may be replaced by a more desirable excipient by use of the separation methods described in the above paragraph. The solvent and first excipient may be discarded with the supernatant after centrifugation or filtration. A fresh volume of the suspension vehicle without the solvent and without the first excipient may then be added. Alternatively, a new surfactant may be added. For example, a suspension consisting of drug, N-methyl-2-pyrrolidinone (solvent), Poloxamer 188 (first excipient), sodium deoxycholate, glycerol and water may be replaced with phospholipids (new surfactant), glycerol and water after centrifugation and removal of the supernatant.

I. First Process Category

The methods of the first process category generally include the step of dissolving the antifungal agent in a water miscible first solvent followed by the step of mixing this solution with an aqueous solution to form a presuspension wherein the antifungal agent is in an amorphous form, a semicrystalline form or in a supercooled liquid form as determined by x-ray diffraction studies, DSC, light microscopy or other analytical techniques and has an average effective particle size within one of the effective particle size ranges set forth above. The mixing step is followed by an energy-addition step and, in a preferred form of the invention is an annealing step.

II. Second Process Category

The methods of the second processes category include essentially the same steps as in the steps of the first processes category but differ in the following respect. An x-ray diffraction, DSC or other suitable analytical techniques of the presuspension shows the antifungal agent in a crystalline form and having an average effective particle size. The antifungal agent after the energy-addition step has essentially the same average effective particle size as prior to the energy-addition step but has less of a tendency to aggregate into larger particles when compared to that of the particles of the presuspension. Without being bound to a theory, it is believed the differences in the particle stability may be due to a reordering of the surfactant molecules at the solid-liquid interface.

III. Third Process Category

The methods of the third category modify the first two steps of those of the first and second processes categories to ensure the antifungal agent in the presuspension is in a friable form having an average effective particle size (e.g., such as slender needles and thin plates). Friable particles can be formed by selecting suitable solvents, surfactants or combination of surfactants, the temperature of the individual solutions, the rate of mixing and rate of precipitation and the like. Friability may also be enhanced by the introduction of lattice defects (e.g., cleavage planes) during the steps of mixing the first solution with the aqueous solution. This would arise by rapid crystallization such as that afforded in the precipitation step. In the energy-addition step these friable crystals are converted to crystals that are kinetically stabilized and having an average effective particle size smaller than those of the presuspension. Kinetically stabilized means particles have a reduced tendency to aggregate when compared to particles that are not kinetically stabilized. In such instance the energy-addition step results in a breaking up of the friable particles. By ensuring the particles of the presuspension are in a friable state, the organic compound can more easily and more quickly be prepared into a particle within the desired size ranges when compared to processing an organic compound where the steps have not been taken to render it in a friable form.

### EXAMPLE 1

### Preparation of 1% Itraconazole Suspension

Each 100 mL of suspension contains:

| | |
|---|---|
| Itraconazole | 1.0 g (1.0% w/v) |
| Deoxycholic Acid, Sodium Salt, Monohydrate | 0.1 g (0.1% w/v) |
| Poloxamer 188, NE | 0.1 g (0.1% w/v) |
| Glycerin, USP | 2.2 g (2.2% w/v) |
| Sodium Hydroxide, NF (0.1 N or 1.0 N) | for pH Adjustment |
| Hydrochloric Acid, NF (0.1 N or 1.0 N) | for pH Adjustment |
| Sterile Water for Injection, USP | QS |
| Target pH (range) | 8.0 (6 to 9) |

Preparation of Surfactant Solution (2 Liters) for Microprecipitation

Fill a properly cleaned tank with Sterile Water for Injection and agitate. Add the required amount of glycerin and stir until dissolution. Add the required amount of deoxycholic acid, sodium salt monohydrate and agitate until dissolution. If necessary, adjust the pH of the surfactant solution with minimum amount of sodium hydroxide and/or hydrochloric acid to a pH of 8.0. Filter the surfactant solution through a 0.2 μm filter. Quantitatively transfer the surfactant solution to the vessel supplying the homogenizer. Chill the surfactant solution in the hopper with mixing.

Preparation of Replacement Solution

Preparation of 4 liters of replacement solution. Fill a properly cleaned tank with WFI and agitate. Add the weighed Poloxamer 188 (Spectrum Chemical) to the measured volume of water. Begin mixing the Poloxamer 188/water mixture until the Poloxamer 188 has completely dissolved. Add the required amount of glycerin and agitate until dissolved. Once the glycerin has completely dissolved, add the required amount of deoxycholic acid, sodium salt monohydrate and stir until dissolution. If necessary, adjust the pH of the wash solution with the minimum amount sodium hydroxide and/or hydrochloric acid to a pH of 8.0. Filter the replacement solution through a 0.2 μm membrane filter.

Preparation of Drug Concentrate

For a 2-L batch, add 120.0 mL of N-methyl-2-pyrrolidinone into a 250-mL beaker. Weigh 2.0 g Poloxamer 188. Weigh 20.0 g of itraconazole (Wyckoff). Transfer the weighed Poloxamer 188 to the 250 mL beaker with N-methyl-2-pyrrolidinone. Stir until dissolved, then add the itraconazole. Heat and stir until dissolved. Cool the drug concentrate to room temperature and filter through a 0.2-micron filter.

Microprecipitation

Add sufficient WFI to the surfactant solution already in the vessel supplying the homogenizer so that the desired target concentration is reached. When the surfactant solution is cooled, start adding the drug concentrate into the surfactant solution with continuous mixing.

Homogenization

Slowly increase the pressure of the homogenizer until the operating pressure 10,000 psi has been reached. Homogenize the suspension with recirculation while mixing. For 2,000 mL of suspension at 50 Hz, one pass should require approximately 54 seconds. Following homogenization, collect a 20-mL sample for particle size analysis. Cool the suspension.

Wash Replacement

The suspension is then divided and filled into 500-mL centrifuge bottles. Centrifuge until clean separation of sediment is observed. Measure the volume of supernatant and replace with fresh replacement solution, prepared earlier. Quantitatively transfer the precipitate from each centrifuge bottle into a properly cleaned and labeled container for resuspension (pooled sample). Resuspension of the pooled sample is performed with a high shear mixer until no visible clumps are observed. Collect a 20-mL sample for particle size analysis.

The suspension is then divided and filled into 500-mL centrifuge bottles. Centrifuge until clean separation of sediment is observed. Measure the volume of supernatant and replace with fresh replacement solution, prepared earlier. Quantitatively transfer the precipitate from each centrifuge bottle into a properly cleaned and labeled container for resuspension (pooled sample). Resuspension of the pooled sample is performed with a high shear mixer until no visible clumps are observed. Collect a 20-mL sample for particle size analysis.

Second Homogenization

Transfer the above suspension to the hopper of the homogenizer and chill the suspension with mixing. Slowly increase the homogenizer pressure until an operating pressure 10,000 psi has been reached. Homogenize while monitoring the solution temperature. Following homogenization, cool the suspension and collect three 30-mL samples for particle analysis. Collect the remaining suspension in a 2-liter bottle.

Filling

Based on acceptable particle size determination testing (mean volume-weighted diameter of 50 nm to 2 microns), collect 30 mL samples in 50 mL glass vials with rubber stoppers.

### EXAMPLE 2

### Other formulations of Itraconazole Suspensions

Other formulations of itraconazole suspensions with different combinations of the surfactants can also be prepared using the method described in Example 1. Table 1 summarizes the compositions of the surfactants of the various itraconazole suspensions.

TABLE 1
Summary of the compositions of the various 1%
itraconazole suspensions

| Formulation No. | Surfactants in the formulation | Amount* |
|---|---|---|
| 1 | Poloxamer 188 | 0.1% |
| | Deoxycholate | 0.1% |
| | Glycerin | 2.2% |
| 2 | Poloxamer 188 | 0.1% |
| | Deoxycholate | 0.5% |
| | Glycerin | 2.2% |
| 3 | Poloxamer 188 | 2.2% |
| | Deoxycholate | 0.1% |
| | Glycerin | 2.2% |
| 4 | Poloxamer 188 | 2.2% |
| | Deoxycholate | 0.5% |
| | Glycerin | 2.2% |
| 9 | Solutol | 0.3% |
| | Deoxycholate | 0.5% |
| | Glycerin | 2.2% |
| 14331-1 | Solutol | 1.5% |
| | Glycerin | 2.2% |
| 14443-1 | Albumin | 5% |

*% by weight of the final volume of the suspension (w/v)

### EXAMPLE 3

### Comparison of the Acute Toxicity Between Commercially Available Itraconazole Formulation (SPORANOX®) and the Suspension Compositions of the Present Invention

The acute toxicity of the commercially available itraconazole formulation (SPORANOX®) is compared to that of the various 1% itraconazole formulations in the present invention as listed in Table 1. SPORANOX® is available from Janssen Pharmaceutical Products, L.P. It is available as a 1% intravenous (I.V.) solution solubilized by hydroxypropyl-β-cyclodextrin. The results are shown in Table 2 with the maximum tolerated dose (MTD) indicated for each formulation.

TABLE 2
Comparison of the acute toxicity of various formulations of itraconazole

| Formulation Number | Results and Conclusions |
|---|---|
| SPORANOX ® I.V. | LD₁₀ = 30 mg/kg |
| | MTD = 20 mg/kg (slight ataxia) |
| 1 | MTD = 320 mg/kg; NOEL = 80 mg/kg |
| | Spleen obs^{b}: 320 mg/kg |
| | Red ears/feet: ≧160 mg/kg |
| 2 | MTD = 320 mg/kg |
| | Spleen obs^{b}: 320 mg/kg |
| | Slight lethargy: 320 mg/kg |
| | Red urine: ≧80 mg/kg |
| | Tail obs^{c}: ≧40 mg/kg |
| 3 | MTD = 160 mg/kg; NOEL = 80 mg/kg |
| | Spleen obs^{b}: 320 mg/kg |
| | Red ears/feet: ≧160 mg/kg |
| 4 | MTD = 160 mg/kg |
| | LD₂₀ = 320 mg/kg |
| | Spleen obs^{b}: 320 mg/kg |
| | Slight lethargy: 320 mg/kg |
| | Red urine: ≧40 mg/kg |
| | Tail obs^{c}: ≧40 m |
| 9 | LD₆₀ = 320 mg/kg; MTD = 160 mg/kg |
| | Spleen obs^{b}: 320 mg/kg |
| | Tail obs: 320 mg/kg |
| | Red ears/feet: ≧160 mg/kg |
| | Red urine: ≧40 mg/kg |
| 14331-1 | MTD = 40 mg/kg; NOEL = 40 mg/kg |
| | LD₄₀ = 80 mg/kg |
| 14443-1 | LD₄₀ = 80 mg/kg; NOEL = 40 mg/kg |

^{a}cyclodextrin = hydroxypropyl-β-cyclodextrin
^{b}Spleen obs = Enlarged and/or pale
^{c}Tail obs = gray to black and/or necrosis
LD₅₀ = Lethal dose resulting in 50% mortality
NOEL = No effect level
MTD = Maximum tolerated dose

### EXAMPLE 4

### Pharmacokinetic Comparison of SPORANOX® vs. Suspension Formulation of Itraconazole

Young adult, male Sprague Dawley rats were treated intravenously (IV) via a caudal tail vein with a single injection at a rate of 1 ml/min. with either SPORANOX® Injection or Formulation 1 at 20 mg/kg. Following administration, the animals were anesthetized and retro-orbital blood was-collected at different time points (n=3). The time points were as follows: 0.03, 0.25, 0.5, 1, 2, 4, 6, 8, 24, 48, 96, 144, 192, 288, and 360 hours (SPORANOX® Injection only to 192 hours). Blood was collected into tubes with EDTA and centrifuged at 3200 rpm for 15 minutes to separate plasma. The plasma was stored frozen at −70° C. until analysis. The concentration of the parent itraconazole and the metabolite hydroxy-itraconazole were determined by high-performance liquid chromatography (HPLC). Pharmacokinetic (PK) parameters for itraconazole (ITC) and hydroxy-itraconazole (OH-ITC) were derived using noncompartmental methods with WinNonlin® Professional Version 3.1 (Pharsight Corp., Mountain View, Calif.).

Table 3 provides a comparison of the plasma pharmacokinetic parameters determined for each itraconazole formulation. Plasma itraconazole was no longer detected at 48 hours for SPORANOX® Injection at 20 mg/kg, and at 96 hours for Formulation 1. Plasma hydroxy-itraconazole was initially detected at 0.25 hours for SPORANOX® Injection and Formulations 1 at 20 mg/kg. Hydroxy-itraconazole was no longer detected at 96 hours for SPORANOX® Injection at 20 mg/kg, and at 144 hours for Formulation 1.

TABLE 3
Comparison of Plasma Pharmacokinetic Parameters for Sporanox and
a Suspension Formulation After IV Administration in Rats

| Analyte | PK Parameters | SPORANOX ® I.V. | Formulation 1 |
|---|---|---|---|
| Itraconazole | Cₘₐₓ (μg/ml) | 13.2 | 30.41 |
| | Tₘₐₓ (h) | 0.03 | 0.03 |
| | AUC (0-∞) | 28.25 | 16.70 |
| | (μg · h/ml) | | |
| | T_{1/2} (h) | 5.36 | 14.36 |
| | CL (μl/h) | 176.97 | 299.35 |
| | MRT (h) | 4.48 | 13.29 |
| Hydroxy- | Cₘₐₓ (μg/ml) | 0.78 | 0.40 |
| itraconazole | Tₘₐₓ (h) | 4.0 | 24 |
| | AUC (0-∞) | 13.41 | 17.89 |
| | (μg · h/ml) | | |
| | T_{1/2} (h) | 5.89 | 15.50 |
| | MRT (h) | 12.17 | 30.99 |

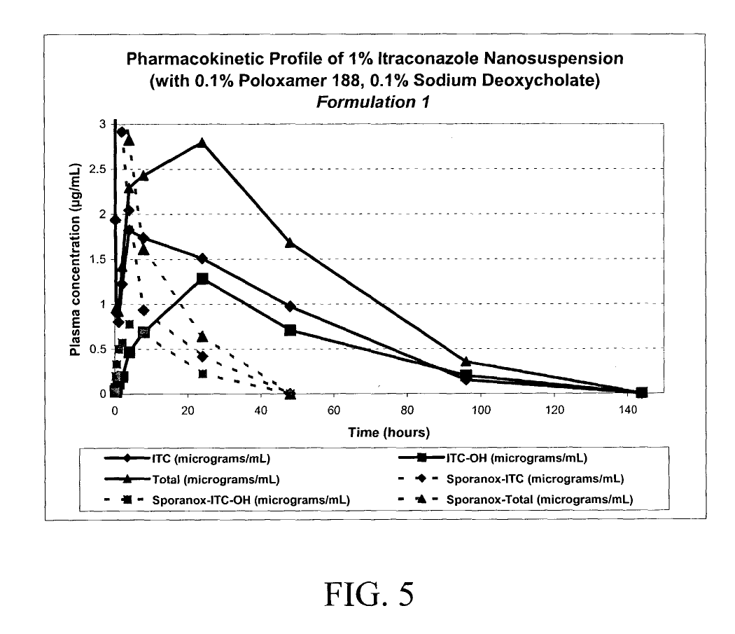

FIG. 5 compares the pharmacokinetics (PK) of SPORANOX® with Formulation 1 suspension of itraconazole particles. Because, as shown above, the present suspension formulation is less toxic than Sporanox®, it was administered at higher amounts in this equitoxic experiment. Sporanox® was dosed at 20 mg/kg and Formulation 1 at 80 mg/kg. The Sporanox® decreases in plasma concentration relatively quickly, over 20 hours. The itraconazole plasma levels remain elevated for approximately 3-4 times longer with the present suspension formulation. The itraconazole exhibits an initial minimum at 30 minutes in the plasma level. This corresponds to a nadir in plasma concentration due to sequestration of the drug nanocrystals by the macrophages of the spleen and liver, thus temporarily removing drug from circulation. However, the drug levels rebound quickly, as the macrophages apparently release the drug into the circulation. Furthermore, the drug with Formulation 1 is metabolized effectively, as is shown by the PK curve for the hydroxy itraconazole metabolite in FIG. 5. The rate of appearance of the metabolite for the suspension formulation is delayed, compared with the PK curve for the metabolite for the SPORANOX® formulation. However, as with the case of the parent molecule for the suspension, the metabolite persists in circulation for a much longer time than is the case with the metabolite for the SPORANOX® formulation. When the AUC (area under the blood concentration vs time curve) is normalized by the dose, the suspension is at least as bioavailable as SPORANOX®.

### EXAMPLE 5

### Pharmacokinetic Studies of Other Suspension Formulations of Itraconazole

Pharmacokinetic studies were also conducted on different formulations of itraconazole at various dosages. The results are summarized in Table 4.

TABLE 4
Plasma Pharmacokinetic Parameters for Various Nanosuspension
Formulations of Itraconazole After IV Administration in Rats

| | | Form- | Form- | Form- |
|---|---|---|---|---|
| | PK | ulation 1, | ulation 1, | ulation 3, |
| Analyte | Parameters | 40 mg/Kg | 80 mg/Kg | 80 mg/Kg |
| Itraconazole | Cₘₐₓ (μg/ml) | 119.16 | 446.33 | 365.09 |
| | Tₘₐₓ (h) | 0.03 | 0.03 | 0.03 |
| | AUC (0-∞) | 42.67 | 143.7 | 108.87 |
| | (μg · h/ml) | | | |
| | T_{1/2} (h) | 23.95 | 25.89 | 38.46 |
| | CL (μl/h) | 234.38 | 139.18 | 183.71 |
| | MRT (h) | 24.37 | 27.45 | 31.21 |
| Hydroxy- | Cₘₐₓ (μg/ml) | 0.61 | 1.03 | 0.52 |
| itraconazole | Tₘₐₓ (h) | 24.0 | 24.0 | 24.0 |
| | AUC (0-∞) | 37.71 | 70.24 | 51.27 |
| | (μg · h/ml) | | | |
| | T_{1/2} (h) | 22.27 | 23.21 | 50.29 |
| | MRT (h) | 43.06 | 46.80 | 60.81 |

### EXAMPLE 6

### Antifungal Efficacy Studies

Normal and immuno-suppressed (prednisolone administered twice daily on the day before and on the day of inoculation) rats inoculated with 9.5×10⁶ or 3×10⁶ cfu C. albicans/ml saline once intravenously were intravenously treated with SPORANOX® Injection once daily for ten consecutive days, with the first dose given 4 to 5 hours after inoculation. SPORANOX® Injection rats were dosed at 5 or 20 mg/kg for the first 2 days, then at 5 or 10 mg/kg for the remaining 8 days, due to toxicity at 20 mg/kg after 2 days of dosing. Similarly, immuno-suppressed rats inoculated with 1×10^{6.5} cfu C. albicans/ml saline were intravenously treated with Formulation 1 at 20, 40, or 80 mg/kg once every other day for ten days, beginning the day of inoculation. The SPORANOX® Injection and Formulation 1 treatment rats were terminated 11 days after the C. albicans inoculation and the kidneys were collected, weighed and cultured for determination of C. albicans colony counts and itraconazole and hydroxy-itraconazole concentration. Kidneys were collected from untreated control rats when a moribund condition was observed or when an animal had a 20% body weight. In addition, body weights were measured periodically during the course of each study.

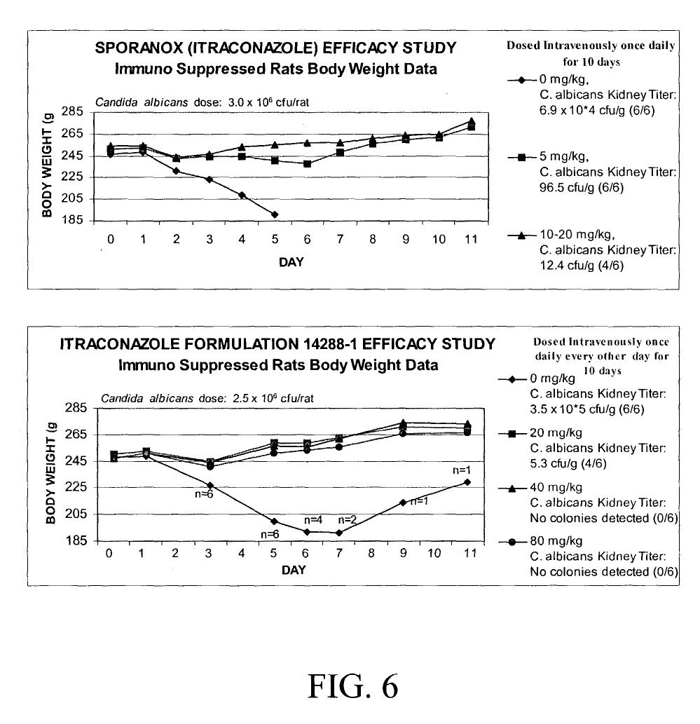

Comparison of results for immuno-suppressed rats treated with SPORANOX® Injection and Formulation 1 are shown in Table 5 and FIG. 6. Daily SPORANOX® Injection treatment at 10-20 mg/kg appeared to be slightly more effective than daily treatment with SPORANOX® Injection at 5 mg/kg. Based on kidney colony counts, every other day dosing at 20 mg/kg of Formulation 1 appeared to be as effective as every day dosing with SPORANOX® Injection at 20 mg/kg and possibly more effective than SPORANOX® Injection at 5 mg/kg (i.e., the recommended clinical dose), whereas the higher doses for Formulation 1 appeared to most effective, based on kidney colony counts (i.e., C. albicans not detected) and increased kidney itraconazole concentration.

TABLE 5
Mean C. albicans Colony Count and Itraconazole and
Hydroxy-Itraconazole Concentration in Kidney

| | | | Concentration in | |
|---|---|---|---|---|
| | C. albicans Titer | | Kidney | |
| | Count | | | OH-ITC |
| Treatment | (cfu/g) | Incidence | ITC (μg/g) | (μg/g) |
| No Treatment | 6.9 × 10⁴ | 6/6 | — | — |
| (3 × 10⁶ cfu/ml) | | | | |
| SPORANOX ®, 5 mg/kg, | 96.5 | 6/6 | 1.2 | 1.5 |
| (3 × 10⁶ cfu/ml) | | | | |
| SPORANOX ®, 10-20 | 12.4 | 4/6 | 8.5 | 8.0 |
| mg/kg, (3 × 10⁶ cfu/ml) | | | | |
| No Treatment | 3.5 × 10⁵ | 6/6 | — | — |
| (2.5 × 10⁶ cfu/ml) | | | | |
| Formulation 1, 20 mg/kg, | 5.3 | 4/6 | 6.1 | 5.7 |
| (2.5 × 10⁶ cfu/ml) | | | | |
| Formulation 1, 40 mg/kg, | 0 | 0/6 | 18.5 | 6.0 |
| (2.5 × 10⁶ cfu/ml) | | | | |
| Formulation 1, 80 mg/kg, | 0 | 0/6 | 41.2 | 6.2 |
| (2.5 × 10⁶ cfu/ml) | | | | |

FIG. 6 is a comparison of the mean body weight and C. albicans colony count data for treatments with SPORANOX® (top panel) and Formulation 1 (bottom panel).

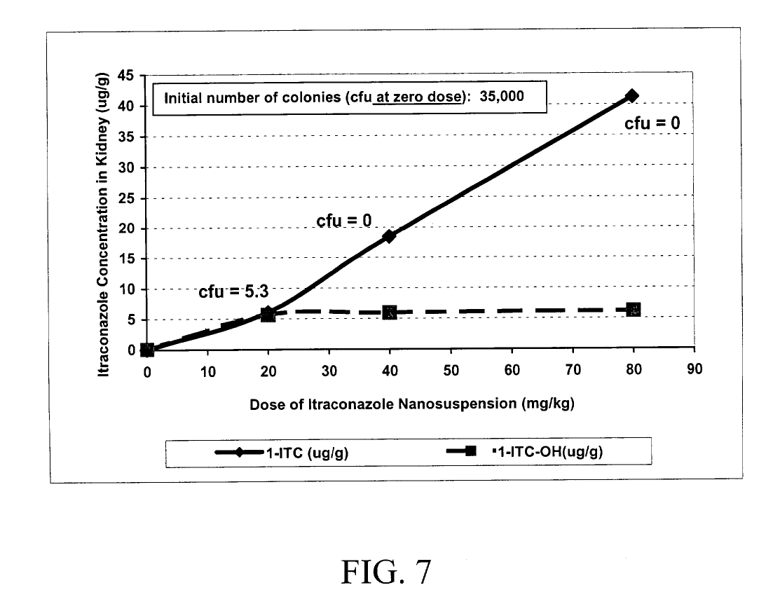

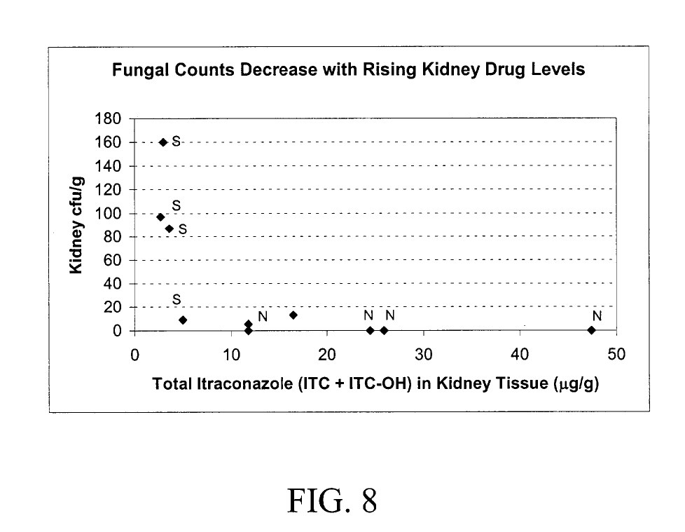

In the examples above, a particulate suspension formulation of an antifungal agent of the present invention was shown to be less toxic than a conventional totally soluble formulation of the same drug. Thus, more of the drug could be administered without eliciting adverse effects. Because the particles of the drug did not immediately dissolve upon injection, they were trapped in a depot store in the liver and spleen. These acted as prolonged release sanctuaries, permitting less frequent dosing. The greater dosing that could be administered permitted greater drug levels to be manifested in the target organs, in this case, the kidney.(FIG. 7). The greater drug levels in this organ led to a greater kill of infectious organisms. (FIG. 8).

### EXAMPLE 7

### Prophetic Examples of Other Triazole Antifungal Agents

The present invention contemplates preparing a 1% suspension of submicron- or micron size of a triazole antifungal agent using the method described in Example 1 and the formulations described in Example 2 with the exception that the antifungal agent is a triazole antifungal agent other than itraconazole. Examples of triazole antifungal agents that can be used include, but are not limited to, ketoconazole, miconazole, fluconazole, ravuconazole, voriconazole, saperconazole, eberconazole, genaconazole, and posaconazole.

### EXAMPLE 8

### Prophetic Example of a Non-Triazole Antifungal Agent

The present invention contemplates preparing a 1% suspension of submicron- or micron size non-triazole antifungal agent using the method described in Example 1 and the formulations described in Example 2 with the exception that the antifungal agent is amphotericin B or flucytosine instead of itraconazole.

From the foregoing, it will be observed that numerous variations and modifications may be effected without departing from the spirit and scope of the invention. It is to be understood that no limitation with respect to the specific apparatus illustrated herein is intended or should be inferred. It is, of course, intended to cover by the appended claims all such modifications as fall within the scope of the claims.

What is claimed is:

1. A composition comprising an aqueous suspension of submicron- to micron-size particles containing an antifungal agent coated with at least one surfactant selected from the group consisting of: ionic surfactants, non-ionic surfactants, biologically derived surfactants, and amino acids and their derivatives, wherein the particles have a volume-weighted mean particle size of less than 50 μm as measured by laser diffractometry.

2. The composition of claim 1, wherein the particles have a volume-weighted mean particle size of less than about 7 μm as measured by laser diffractometry.

3. The composition of claim 1, wherein the particles have a volume-weighted mean particle size of less than about 2 μm as measured by laser diffractometry.

4. The composition of claim 1, wherein the particles have a volume-weighted mean particle size of less than about 400 nm as measured by laser diffractometry.

5. The composition of claim 1, wherein the particles have a volume-weighted mean particle size of less than 100 nm as measured by laser diffractometry.

6. The composition of claim 1, wherein the antifungal agent is a triazole antifungal agent.

7. The composition of claim 6, wherein the triazole antifungal agent is selected from the group consisting of: itraconazole, ketoconazole, miconazole, fluconazole, ravuconazole, voriconazole, saperconazole, eberconazole, genaconazole, and posaconazole.

8. The composition of claim 1, wherein the antifungal agent is itraconazole.

9. The composition of claim 1, wherein the ionic surfactant is selected from the group consisting of: anionic surfactants and cationic surfactants.

10. The composition of claim 9, wherein the anionic surfactant is selected from the group consisting of: potassium laurate, triethanolamine stearate, sodium lauryl sulfate, sodium dodecylsulfate, alkyl polyoxyethylene sulfates, sodium alginate, dioctyl sodium sulfosuccinate, glyceryl esters, sodium carboxymethylcellulose, bile acids and their salts, and calcium carboxymethylcellulose.

11. The composition of claim 10, wherein the bile acid is selected from the group consisting of cholic acid, deoxycholic acid, glycocholic acid, taurocholic acid, and glycodeoxycholic acid.

12. The composition of claim 9, wherein the cationic surfactant is selected from the group consisting of quaternary ammonium compounds, benzalkonium chloride, cetyltrimethylammonium bromide, chitosans and lauryldimethylbenzylammonium chloride.

13. The composition of claim 1, wherein the nonionic surfactant is selected from the group consisting of: polyoxyethylene fatty alcohol ethers, sorbitan fatty acid esters, polyoxyethylene fatty acid esters, sorbitan esters, glycerol monostearate, polyethylene glycols, cetyl alcohol, cetostearyl alcohol, stearyl alcohol, poloxamers, poloxamines, methylcellulose, hydroxycellulose, hydroxy propylcellulose, hydroxy propylmethylcellulose, noncrystalline cellulose, polyvinyl alcohol, and polyvinylpyrrolidone.

14. The composition of claim 1, wherein the biologically derived surfactant is selected from the group consisting of: albumin, heparin, casein and hirudin.

15. The composition of claim 1, wherein the amino acid is selected from the group consisting of: leucine, alanine, valine, isoleucine, lysine, aspartic acid, glutamic acid, methionine, and phenylalanine.

16. The composition of claim 1, wherein the amino acid derivative is an amide, an ester, or a polypeptide.

17. The composition of claim 1, wherein the surfactant is a bile salt.

18. The composition of claim 17, wherein the bile salt is deoxycholate.

19. The composition of claim 1, wherein the surfactant is a polyalkoxyether.

20. The composition of claim 19, wherein the polyalkoxyether is Poloxamer 188.

21. The composition of claim 1, wherein the surfactant is hydroxyethylstarch.

22. The composition of claim 1, wherein the surfactant is polyethylene-660-hydroxystearate.

23. The composition of claim 1, wherein the surfactant is albumin.

24. The composition of claim 1, wherein the aqueous medium further comprises a pH adjusting agent.

25. The composition of claim 24, wherein the pH adjusting agent is selected from the group consisting of: tris buffer, phosphate, acetate, lactate, tris(hydroxymethyl)aminomethane, meglumine (N-methylglucosamine), citrate, sodium hydroxide, hydrochloric acid, and amino acids.

26. The composition of claim 25, wherein the amino acid is selected from the group consisting of: glycine, arginine, lysine, alanine, and leucine.

27. The composition of claim 1, further comprising an osmotic pressure adjusting agent.

28. The composition of claim 27, wherein the osmotic pressure adjusting agent is selected from the group consisting of: glycerin, monosaccharides, and sugar alcohols.

29. The composition of claim 28, wherein the monosaccharide is dextrose.

30. The composition of claim 28, wherein the sugar alcohol is mannitol or sorbitol.

31. The composition of claim 1, wherein the antifungal agent is present is an amount of from about 0.01% to about 50% w/v.

32. The composition of claim 1, wherein the antifungal agent is present in an amount of from about 0.05% to about 30% w/v.

33. The composition of claim 1, wherein the antifungal agent is present in an amount of about 0.1 % to about 20% w/v.

34. The composition of claim 1, wherein the surfactant is present in an amount of from about 0.001% to about 5% W/V.

35. The composition of claim 1, wherein the surfactant is present in an amount of from about 0.005% to about 5% W/V.

36. The composition of claim 1, wherein the surfactant is present in an amount of from about 0.01% to about 5% W/V.

37. The composition of claim 1 is administered by a route selected from the group consisting of: parenteral, oral, buccal, periodontal, rectal, nasal, pulmonary, and topical.

38. The composition of claim 1 is administered by parenteral administration.

39. The composition of claim 38, wherein the parenteral administration is selected from the group consisting of: intravenous, intra-arterial, intrathecal, intraperitoneal, intraocular, intra-articular, intramuscular, and subcutaneous injection.

40. The composition of claim 1, wherein the aqueous medium is removed to form dry particles.

41. The composition of claim 40, wherein the method of removing the aqueous medium is selected from the group consisting of: evaporation and lyophilization.

42. The composition of claim 40, wherein the method of removing the aqueous medium is by lyophilization.

43. The composition of claim 40, wherein the dry particles are formulated into an acceptable pharmaceutical dosage form.

44. The composition of claim 43, wherein the pharmaceutical dosage form is selected from the group consisting of: parenteral solutions, tablets, capsules, suspensions, creams, lotions, emulsions, pulmonary formulations, topical formulations, controlled or sustained release formulations, and tissue specific targeted delivery formulations.

45. The composition of claim 1, wherein the composition is frozen.

46. A composition comprising an aqueous suspension of submicron- to micron-size particles of itraconazole coated with at least one surfactant, and an osmotic pressure adjusting agent, wherein the nanoparticles having a volume-weighted mean particle size of less than 50 μm as measured by laser diffractometry, and wherein the itraconazole is present in an amount of from about 0.01% to about 50% w/v, and the surfactant is present in an amount of from about 0.001% to about 5%.

47. The composition of claim 46, wherein the surfactant is selected from the group consisting of: bile salts, polyalkoxyethers, hydroxytheylstarch, polyethylene-660-hydroxystearate, and albumin.

48. The composition of claim 47, wherein the bile salt is deoxycholate.

49. The composition of claim 47, wherein the polyalkoxyether is Poloxamer 188.

50. The composition of claim 46, wherein the surfactant is hydroxyethylstarch.

51. The composition of claim 46, wherein the surfactant is polyethylene-660-hydroxystearate.

52. The composition of claim 46, wherein the surfactant is albumin.

53. The composition of claim 46, wherein the osmotic pressure adjusting agent is glycerin.

54. The composition of claim 46, wherein the particles having a volume-weighted mean particle size of less than 7 μm as measured by laser diffractometry.

55. The composition of claim 46, wherein the particles having a volume-weighted mean particle size of less than 2 μm as measured by laser diffractometry.

56. The composition of claim 46, wherein the particles having a volume-weighted mean particle size of less than 400 nm as measured by laser diffractometry.

57. The composition of claim 46, wherein the particles having a volume-weighted mean particle size of less than 100 nm as measured by laser diffractometry.

58. A composition comprising an aqueous suspension of submicron- to micron-size particles of itraconazole coated with at least one surfactant, and an osmotic pressure adjusting agent, wherein the particles having a volume-weighted mean particle size of less than 2 μm as measured by laser diffractometry, the surfactant is selected from the group consisting of: bile salts, polyalkyoxyethers, hydroxytheylstarch, polyethylene-660-hydroxystearate, and albumin, the itraconazole is present in an amount of from about 0.01% to about 50% w/v, and the surfactant is present in an amount of from about 0.001% to about 5%.

59. The composition of claim 58, wherein the osmotic pressure adjusting agent is glycerin.

60. A composition comprising an aqueous suspension of submicron- to micron-size particles of itraconazole coated with a mixture of surfactants comprising a bile salt and a polyalkoxyether, and glycerin as an osmotic pressure adjusting agent, wherein the particles having a volume-weighted mean particle size of less than about 2 μm as measured by laser diffractometry, and wherein the itraconazole is present in an amount of from about 0.01% to about 50% w/v, bile salt is present in an amount of from about 0.001% to about 5% w/v, the polyalkoxyether is present in an amount of from about 0.001% to about 5% w/v, and glycerin is present in an amount of about 2.2% w/v.

61. The composition of claim 60, wherein the bile salt is deoxycholate.

62. The composition of claim 60, wherein the polyalkyoxyether is Poloxamer 188.

63. A composition comprising an aqueous suspension of submicron-to micron-size particles of itraconazole coated with a mixture of surfactants comprising a bile salt, and polyethylene-660-hydroxystearate, and glycerin as an osmotic pressure adjusting agent, wherein the particles having a volume-weighted mean particle size of less than 2 μm as measured by laser diffractometry, and wherein itraconazole is present in an amount of from about 0.01% to about 50% w/v, the bile salt is present in an amount from about 0.001% to about 5% w/v, polyethylene-660-hydroxystearate is present in an amount of from about 0.001% to about 5% w/v, and glycerin is present in an amount of about 2.2% w/v.

64. A composition of particles of an antifungal agent prepared by a method comprising the steps of:
(i) dissolving the antifungal agent in a water-miscible first solvent to form a solution, the first solvent being selected from the group consisting of N-methyl-2-pyrrolidinone, 2-pyrrolidone, dimethyl sulfoxide, dimethylacetamide, lactic acid, acetic acid and other liquid carboxylic acids, methanol, ethanol, isopropanol, 3-pentanol, n-propanol, glycerol, butylene glycol, ethylene glycol, propylene glycol, mono- and diacylated monoglycerides, dimethyl isosorbide, acetone, dimethylformamide, 1,4-dioxane, polyethylene glycol, polyethylene glycol esters, polyethylene glycol sorbitans, polyethylene glycol monoalkyl ethers, polypropylene glycol, polypropylene alginate, PPG-10 butanediol, PPG-10 methyl glucose ether, PPG-20 methyl glucose ether, PPG-15 stearyl ether, propylene glycol dicaprylate, propylene glycol dicaprate, propylene glycol laurate;
(ii) mixing the solution with a second solvent which is aqueous to define a pre-suspension; and
(iii) adding energy to the pre-suspension to form particles having an average effective particle size of less than 50 μm;
wherein the solubility of the antifungal agent is greater in the first solvent than in the second solvent, and the second solvent comprising one or more surfactants selected from the group consisting of: nonionic surfactants, ionic surfactants, biologically derived surfactants, and amino acids and their derivatives.

65. The composition of claim 64, wherein the average effective particle size is less than about 7 μm.

66. The composition of claim 64, wherein the average effective particle size is less than about 2 μm.

67. The composition of claim 64, wherein the average effective particle size is less than about 400 nm.

68. The composition of claim 64, wherein the average effective particle size is less than about 100 nm.
